(19) United States
(12) Patent Application Publication
(10) Pub. No.: US 2024/0076461 A1
(43) Pub. Date: Mar. 7, 2024

(54) COATED CELLULOSE-BASED SUBSTRATE

(71) Applicant: Stora Enso OYJ, Helsinki (FI)

(72) Inventors: Gisela CUNHA, Nacka (SE); Carl-Magnus Brandèn, Karlstad (SE); Adeel Shahzad, Huddinge (SE); Mikael Gällstedt, Stockholm (SE)

(73) Assignee: Stora Enso OYJ, Helsinki (FI)

(21) Appl. No.: 18/260,812

(22) PCT Filed: Jan. 20, 2022
(86) PCT No.: PCT/IB2022/050456

§ 371 Date: Jul. 10, 2023

(30) Foreign Application Priority Data
Jan. 21, 2021 (SE) 2150063-2

(51) Int. Cl. C08J 7/04; B29C 71/02; C08J 7/02; C08J 7/12; C09D 101/02

(52) U.S. Cl. CPC C08J 7/04; C08J 7/02; C08J 7/08; C08J 7/12; C09D 101/02; C08J 2301/02

(57) ABSTRACT

The present invention relates to a coated cellulose-based substrate, comprising a cellulose-based substrate, and a coating layer disposed on said cellulose-based substrate, wherein said coating layer comprises regenerated cellulose and wherein said coating layer has been hydrophobized with a hydrophobizing agent. The invention further relates to a method for manufacturing the coated cellulose-based substrate.

### TECHNICAL FIELD

The present disclosure relates to coating of cellulose-based substrates to improve the barrier properties, particularly the liquid repellency, of the substrate.

### BACKGROUND

Liquid repellency, and particularly water-resistance, is an important property in many paper or paperboard applications. Some examples include packaging, such as boxes, and other containers; fresh and aseptic liquid packaging; boxes, trays, or cups for hot, cold, dry, wet and frozen food and beverages; products for outdoor use such as boxes, signs and posters; pots, trays and covers for plants; packages for construction materials, and construction materials.

Coating of paper or paperboard with plastics is often employed to combine the mechanical properties of the paperboard with the barrier and sealing properties of a plastic film. A problem with the addition of plastics is that the repulpability of the material is severely reduced, which also can affect the recycling streams. Therefore, as few different material types as possible is desirable in packaging materials.

Furthermore, in order to reduce the carbon footprint and increase the recyclability of cellulose-based products it is of general interest to replace the fossil-based synthetic polymers often used for improving liquid repellency today, with biobased materials. However, it has been found difficult to achieve an acceptable combination of properties, including water resistance or water repellency, durability and feel, using biobased components.

In recent years, grafting with fatty acid halides has emerged as a method for rendering cellulose-based substrates hydrophobic. This technique utilizes fatty acid halides, preferably fatty acid chlorides, which are grafted covalently to hydroxyl groups on cellulose-based substrates, like paper and paperboard. The grafting is performed on the pre-formed and dried material. The fatty acid halide reagent can be applied in liquid, gas and/or spray form to the surface of the substrate. The reagent typically diffuses to a certain depth, typically around 100-150 μm, depending on the density of the substrate and the production speed. Grafting with fatty acid halides is a promising bio-based and sustainable technology to provide water barrier properties to cellulosic materials, but it usually requires a smooth surface for best results.

A rough surface might not only lead to inefficient fatty acid halide grafting treatment, but also might have other negative aspects, such as poor adhesion to laminate layers and poor printability, difficult to coat with thin layer coatings, such as AlOx, SiOx and metallization coatings. A rough surface might also lead to accumulation of dirt and dust, and to other issues related to friction and wear.

Thus, there remains a need in the industry for improved solutions for rendering cellulose-based products water resistant or water repellent, which are based on biobased materials. Such solutions could reduce the need for fossil-based plastics in the production of cellulose-based products, which is beneficial both from a sustainability and recyclability perspective.

### DESCRIPTION OF THE INVENTION

It is an object of the present disclosure to provide a method for rendering cellulose-based substrates liquid repellent, which alleviates at least some of the above-mentioned problems.

It is a further object of the present disclosure to provide a method for rendering cellulose-based substrates liquid repellent, which uses mainly or only biobased materials.

It is a further object of the present disclosure to provide liquid repellent coating for a cellulose-based substrate, wherein the coating is based mainly or only on biobased materials.

The above-mentioned objects, as well as other objects as will be realized by the skilled person in the light of the present disclosure, are achieved by the various aspects of the present disclosure.

The inventors have found that coating of cellulose-based substrates with a regenerated cellulose (RC) and hydrophobizing the RC coated cellulose-based substrate with a hydrophobizing agent provides a hydrophobic coated cellulose-based substrate with improved water barrier properties, improved overall barrier properties at high relative humidities (RH) and shows enhanced dimensional stability. It is further realized that this type of coating may also be applied to other substrates that would benefit from such improved properties. Particularly, it is realized that the coating may be advantageously applied to cellulose-based substrates, such as paper and paperboard to improve the performance of such substrates in various applications.

Advantages of the inventive coated substrates compared to uncoated substrates include improved water repellency, improved oil repellency, adjustable stiffness, and reduced surface roughness.

According to a first aspect illustrated herein, there is provided a coated cellulose-based substrate, comprising
a cellulose-based substrate, and
a coating layer disposed on said cellulose-based substrate,
wherein said coating layer comprises regenerated cellulose and wherein said coating layer has been hydrophobized with a hydrophobizing agent.

The cellulose-based substrate is preferably a web or sheet having a first main surface and a second main surface. The coating layer can be disposed on one or both of the main surfaces, depending on the intended use of the coated substrate.

The present invention is based on the surprising finding that a cellulose-based substrate can be provided with a coating of regenerated cellulose (RC) and subsequently hydrophobized with a hydrophobizing agent to provide a hydrophobic coating with improved water repellency, improved oil repellency, adjustable stiffness, and reduced surface roughness.

Regenerated cellulose is a colorless, transparent and non-toxic pure cellulose product. It is produced from wood pulp, which is dissolved with a specific solvent or solvent system to obtain a cellulose solution. The solution can subsequently be used for forming regenerated cellulose fibers or films. Examples of regenerated cellulose fibers include Rayon and Lyocell. An example of a regenerated cellulose film is Cellophane. The regenerated cellulose may be obtained from any suitable cellulose source. In some embodiments, the regenerated cellulose is obtained from dissolving pulp, also referred to as dissolving cellulose. Dissolving pulp is bleached wood pulp or cotton linters that has a high cellulose content (>90%). It has special properties including a high level of brightness and uniform molecular-weight distribution. Dissolving pulp is manufactured for uses that require a high chemical purity, and particularly low hemicellulose content, since the chemically similar hemicellulose can interfere with subsequent processes. Dissolving pulp is so named because it is not made into paper, but dissolved either in a solvent or by derivatization into a homogeneous solution, which makes it completely chemically accessible and removes any remaining fibrous structure. Once dissolved, the dissolving pulp can be spun into textile fibers or formed into films.

Coating of the cellulose-based substrate with a coating comprising regenerated cellulose can be achieved by at least partially dissolving cellulose in a solvent or solvent system capable of dissolving the cellulose, applying the formed cellulose solution onto the cellulose-based substrate, and regenerating the dissolved cellulose to obtain a coating comprising the regenerated cellulose on the substrate surface.

Examples of solvents and derivatizing solvent systems useful for dissolving cellulose include, but are not limited to, an NaOH solution, N-methylmorpholine N-oxide (NMMO), ionic liquids, urea/NaOH solution, thiourea/urea/NaOH solution, and NaOH/carbon disulfide (CS₂). In the majority of the cases, cellulose is not dissolved down to a molecular level but rather forms stable colloidal dispersions where ordered cellulose aggregates of, at least, several hundred chains, are present. The term cellulose solution as used herein is therefore intended to include solutions of fully dissolved cellulose as well as stable colloidal dispersions comprising partially dissolved cellulose.

The concentration of cellulose in the cellulose solution may be selected depending on the intended coating technique and desired coating layer thickness. The concentration of cellulose in the cellulose solution may be in the range of 0.01-20 wt %, preferably in the range of 0.1-10 wt %.

Application of the cellulose solution onto the cellulose-based substrate may be done by various coating techniques known in the art. Examples include, but are not limited to, dip coating, curtain coating, roll coating, blade coating and spray coating.

The coating may be provided on one or more surfaces of the cellulose-based substrate. The coating can be achieved by applying the cellulose solution to one or both sides of the cellulose-based substrate. The coating can also be achieved by impregnating the cellulose-based substrate with the cellulose solution.

Regeneration of the dissolved cellulose can be achieved by subjecting the cellulose solution to a treatment with a coagulation medium. The coagulation medium is selected depending on the solvent or solvent system used. Suitable combinations of solvents and coagulation media are known in the art. In some embodiments, the regeneration of the dissolved cellulose is achieved by treating the cellulose solution with an acidic solution, preferably a sulfuric acid solution.

The regenerated cellulose may be subjected to one or more washing steps to remove solvent residues and other contaminants.

The regenerated cellulose may be subjected to treatment with a plasticizer. The plasticizer may for example be glycerol.

Finally, the RC coated cellulose-based substrate is dried. The term “dry” as used herein means that the coated cellulose-based substrate has a dry content above 80%, preferably above 90%, and more preferably above 95% by weight.

The cellulose-based substrate is preferably a web or sheet having a first main surface and a second main surface. The coating layer can be disposed on one or both of the main surfaces, depending on the intended use of the coated substrate.

In some cases, the cellulose-based substrates will have an RC coating layer on one of its two main surfaces, whereas the other of the two main surfaces is uncoated. Such one-side RC coated substrates can advantageously be subjected to hydrophobization on the coated surface as well as on the uncoated surface. Thus, in some embodiments, wherein no RC coating layer is disposed on the second main surface of the substrate, the uncoated surface has also been subjected to hydrophobization.

The coating of the cellulose-based substrate with regenerated cellulose reduces the pore size and the permeability of the substrate. Accordingly, the coated substrate will typically have a lower air permeability then the uncoated cellulose-based substrate.

The coating layer may be comprised entirely of the regenerated cellulose, or it can comprise a mixture of the regenerated cellulose and other ingredients or additives. The coating layer preferably includes regenerated cellulose as its main component based on the total dry weight of the coating layer. In some embodiments, the coating layer comprises at least 50 wt %, preferably at least 70 wt %, and more preferably at least 90 wt % of regenerated cellulose, based on the total dry weight of the coating layer. In a preferred embodiment, the coating layer comprises at least 95 wt %, preferably at least 98 wt % of regenerated cellulose, based on the total dry weight of the coating layer.

In some embodiments, the dry basis weight of the coating layer is in the range of 0.1-30 gsm, preferably in the range of 1-25 gsm, more preferably in the range of 2-gsm.

The inventors have further found that the RC coated cellulose-based substrate can be hydrophobized with a hydrophobizing agent to provide a hydrophobic coating. In some embodiments, the hydrophobizing agent may also render the RC coated cellulose-based substrate oleophobic. A surface which is both hydrophobic and oleophobic may also be referred to as being omniphobic. Examples of hydrophobizing agents that can render the RC coated cellulose-based substrate omniphobic are fluorinated hydrophobizing agents.

The terms hydrophobic, hydrophobized and hydrophobizing as used herein generally refer to surfaces that exhibit an apparent water contact angle that is greater than 90°, or to methods for modifying a surface to impart an apparent water contact angle that is greater than 90°.

The term oleophobic generally refers to surfaces that exhibit an apparent contact angle with hexadecane that is greater than 90°.

In order to reduce the carbon footprint and increase the recyclability of cellulose products it is of general interest to replace the synthetic polymers often used for improving liquid repellency today, with biobased materials. Thus, in preferred embodiments the hydrophobizing agent used for hydrophobizing the RC coated cellulose-based substrate is based on biobased materials. Such solutions could reduce the need for fossil-based plastics in the production of cellulose-based packaging materials, which is beneficial both from a sustainability and recyclability perspective. Using a hydrophobizing agent based on biobased materials allows for the entire coated cellulose-based substrate to be biobased. Thus, in some embodiments, the hydrophobizing agent is biobased. In some embodiments, the entire coated cellulose-based substrate is biobased.

The hydrophobizing agent should preferably be selected so as to provide good adhesion to the RC coated cellulose-based substrate. The hydrophobizing agent can be applied to RC coated surfaces only, or to both RC coated and uncoated surfaces of the cellulose-based substrate.

The hydrophobizing agent is preferably covalently bound to the regenerated cellulose. A covalently bound hydrophobizing agent is preferred since it will generally have higher adhesion to the RC coated substrate than a non-covalently bound hydrophobizing agent.

In some preferred embodiments, the hydrophobizing agent is a hydrophobizing agent capable of being covalently grafted to the regenerated cellulose.

Thus, in some embodiments, the hydrophobizing agent has
i) a hydrophobic portion and
ii) a reactive portion capable of forming a covalent bond to the regenerated cellulose.

In some preferred embodiments, the hydrophobizing agent is selected from the group consisting of fatty acid halides, fatty acid anhydrides, epoxidized fatty acids, and combinations thereof.

In some preferred embodiments, the hydrophobizing agent is a fatty acid halide, preferably a fatty acid halide having an aliphatic chain length of 8-22 carbon atoms. Examples of fatty acid halides include octanoyl chloride (C8), lauroyl chloride (C12), myristoyl chloride (C14), palmitoyl chloride (C16), and stearoyl chloride (C18), and/or a mixture thereof. In some preferred embodiments, the fatty acid halide grafted on the RC coated substrate is palmitoyl chloride or stearoyl chloride.

The coated substrate surface has a high content of cellulose molecules. Grafting with fatty acid halide has been identified as an interesting alternative for rendering cellulose-based substrates hydrophobic. The high reactivity of the fatty acid halides results in that the reagent will be covalently bound to the substrate to a high extent, reducing issues related to leaching and migration.

Grafting of the fatty acid halide to the RC coated substrate having available hydroxyl groups can be achieved by applying a fatty acid halide to the surface of the coating, followed by penetration of the reagent upon heating, which also promotes the formation of covalent bonds between the fatty acid halide and the hydroxyl groups of the regenerated cellulose. The fatty acid halide grafting is preferably performed on the RC coated and dried cellulose-based substrate. The grafting typically involves contacting the RC coated substrate with a fatty acid halide in a liquid, spray and/or vapor state. The reaction between the fatty acid halide, e.g. fatty acid chloride, and the hydroxyl groups of the regenerated cellulose results in ester bonds between the reagent and the cellulose. Ungrafted and thereby unbound fatty acids may also be present to a certain extent. Upon the reaction with the hydroxyl groups in the regenerated cellulose, and/or with water in the substrate and/or in the air, hydrohalic acid, e.g. hydrochloric acid, is formed as a reaction byproduct. The grafting may preferably be followed by removal of the formed hydrohalic acid, and optionally by removal of the ungrafted residues. One example of a grafting process which could be used in production of the coated cellulose-based substrate of the present disclosure is described in detail in the international patent application WO2012066015A1. Another example of a grafting process, which could be used in production of the coated cellulose-based substrate in the present disclosure, is described in detail in the international patent application WO2017002005A1. The grafting process may also be repeated, in order to increase the amount of grafted and free fatty acids in the coating.

The coated cellulose-based substrate is preferably dry when the fatty acid halide grafting is performed. The term “dry” as used herein means that the coated cellulose-based substrate has a dry content above 80%, preferably above 90%, and more preferably above 95% by weight.

The fatty acid halide grafting preferably results in a total amount of grafted and free fatty acids in the coated cellulose-based substrate in the range of 0.05-5 kg/ton of the total dry weight of the coated cellulose-based substrate.

Advantages of the inventive coated substrate compared to an uncoated substrate include improved water repellency, improved oil repellency, improved wind resistance, adjustable stiffness, and reduced surface roughness.

The coated cellulose-based substrate is preferably water repellent. The term “water repellent” generally means that the coated cellulose-based substrate has a higher resistance to water absorption than the same cellulose-based substrate without the coating.

In some embodiments, a surface of said coating layer treated with the hydrophobizing agent has a water contact angle above 90°, preferably above 100°.

In some embodiments, the cellulose-based substrate is a fibrous cellulose-based substrate.

The cellulose-based substrate (also referred to herein as “the substrate”) is preferably a sheet or web of material mainly formed from pulp of wood or other fibrous substances comprising cellulose fibers. In some embodiments, the cellulose-based substrate is selected from the group consisting of paper, paperboard, a bacterial cellulose membrane, and a microfibrillated cellulose film, or a combination thereof. The cellulose-based substrate is preferably paper or paperboard.

Paper generally refers to a material manufactured in sheets or rolls from the pulp of wood or other fibrous substances comprising cellulose fibers, used for e.g. writing, drawing, or printing on, or as packaging material. Paper can either be bleached or unbleached and produced in a variety of thicknesses, depending on the end-use requirements.

Paperboard generally refers to strong, thick paper or cardboard comprising cellulose fibers used for example as flat substrates, trays, boxes and/or other types of packaging. Paperboard can either be bleached or unbleached and produced in a variety of thicknesses, depending on the end-use requirements.

In some embodiments, the cellulose-based substrate is comprised of two or more cellulose-based plies. Each of the cellulose-based plies can have a certain composition of pulp fibers, such as bleached and/or unbleached Kraft pulp, sulfite pulp, dissolving pulp, thermomechanical pulp (TMP), chemi-thermomechanical pulp (CTMP), high-temperature CTMP (HT-CTMP) and/or mixtures thereof. The different plies can have different grammages and/or thicknesses and may contain different amounts of internal sizing agent and/or grafted fatty acids.

As an example, the substrate can be built up of one top-ply consisting of bleached Kraft pulp, a mid-ply consisting of a mixture of bleached Kraft pulp and CTMP, and a bottom-ply consisting of bleached Kraft pulp, wherein the mid-ply has a higher thickness than both the top and bottom plies, respectively.

In some embodiments, the basis weight of the cellulose-based substrate is in the range of 20-800 g/m².

In some embodiments, the coated cellulose-based substrate additionally comprises a thin coating layer disposed on the hydrophobized coating layer, said thin coating layer having a thickness in the range of 0.1-100 nm. The thin coating layer may for example be a metal or ceramic or organic coating applied on the hydrophobized coating layer. The coated cellulose-based substrate will typically have a smoother surface than the corresponding uncoated substrate. The increased smoothness makes the surface more suitable for evenly applying thin coatings, such as AlOx, SiOx and metallization coatings.

In some embodiments, a surface of said coating layer treated with the hydrophobizing agent has a water contact angle above 90°, preferably above 100°.

In some embodiments, a surface of said coating layer treated with the hydrophobizing agent has a Cobb₆₀ value (as determined according to standard ISO 535:2014 after 60 seconds) below 20 g/m², preferably below 15 g/m², more preferably below 10 g/m².

In some embodiments, the coated cellulose-based substrate has a repulpability characterized by a reject rate (as determined according to the PTS RH 021/97 test method) below 20%, preferably below 10%, and more preferably below 5%.

According to a second aspect illustrated herein, there is provided a method for manufacturing a coated cellulose-based substrate, said method comprising:
a) providing a cellulose-based substrate,
b) coating the cellulose-based substrate with a coating layer comprising a regenerated cellulose, and
c) hydrophobizing the coating layer with a hydrophobizing agent.

In some embodiments, the coating layer is formed by applying cellulose dissolved in a solvent onto the cellulose-based substrate and treating the dissolved cellulose with a coagulation medium to obtain the coating layer comprising regenerated cellulose (also referred to herein as “RC coating”).

In some embodiments, the solvent is a non-derivatizing solvent selected form the group consisting of an alkaline aqueous solution, N-methylmorpholine N-oxide (NMMO), and an ionic liquid.

In some embodiments, the solvent is a derivatizing solvent system selected form the group consisting of urea and NaOH solution, thiourea, urea, and NaOH solution, and NaOH and carbon disulfide (CS₂).

The concentration of cellulose in the cellulose solution may be in the range of 0.01-20 wt %, preferably in the range of 0.1-10 wt %.

Application of the cellulose solution onto the cellulose-based substrate may be done by various coating techniques known in the art. Examples include, but are not limited to, dip coating, curtain coating, roll coating, blade coating and spray coating.

The RC coating may be provided on one or more surfaces of the cellulose-based substrate. The coating can be achieved by applying the cellulose solution to one or both sides of the cellulose-based substrate. The coating can also be achieved by impregnating the cellulose-based substrate with the cellulose solution.

Regeneration of the dissolved cellulose can be achieved by subjecting the cellulose solution to a treatment with a coagulation medium. The coagulation medium is selected depending on the solvent or solvent system used. Suitable combinations of solvents and coagulation media are known in the art. In some embodiments, the regeneration of the dissolved cellulose is achieved by treating the cellulose solution with an acidic solution, preferably a sulfuric acid solution.

The RC coating may be subjected to one or more washing steps to remove solvent residues and other contaminants.

The RC coating may further be subjected to treatment with a plasticizer. The plasticizer may for example be glycerol.

Finally, the RC coated cellulose-based substrate is dried. The term “dry” as used herein means that the coated cellulose-based substrate has a dry content above 80%, preferably above 90%, and more preferably above 95% by weight. Thus, the coating step b) preferably further comprises drying the coated cellulose-based substrate, preferably to a dry content above 80%, more preferably above 90%, and more preferably above 95% by weight.

In some embodiments, the dry basis weight of the coating layer is in the range of 0.1-30 gsm, preferably in the range of 1-25 gsm, more preferably in the range of 2-15 gsm.

The hydrophobizing agent should preferably be selected so as to provide good adhesion to the RC coated cellulose-based substrate. The hydrophobizing agent can be applied to RC coated surfaces only, or to both RC coated and uncoated surfaces of the cellulose-based substrate

The hydrophobizing agent is preferably covalently bound to the regenerated cellulose. A covalently bound hydrophobizing agent is preferred since it will generally have better adhesion to the RC coated substrate than a non-covalently bound hydrophobizing agent.

In some preferred embodiments, the hydrophobizing agent is a hydrophobizing agent capable of being covalently grafted to the regenerated cellulose.

Thus, in some embodiments, the hydrophobizing agent has
i) a hydrophobic portion and
ii) a reactive portion capable of forming a covalent bond to the regenerated cellulose.

In some preferred embodiments, the hydrophobizing agent is selected from the group consisting of fatty acid halides, fatty acid anhydrides, epoxidized fatty acids, epoxidized fatty acid (methyl) esters, and combinations thereof.

In some preferred embodiments, the hydrophobizing agent is a fatty acid halide, preferably a fatty acid halide having an aliphatic chain length of 8-22 carbon atoms. Examples of fatty acid halides include octanoyl chloride (C8), lauroyl chloride (C12), myristoyl chloride (C14), palmitoyl chloride (C16), and stearoyl chloride (C18), and/or a mixture thereof. In some preferred embodiments, the fatty acid halide grafted on the RC coated substrate is palmitoyl chloride or stearoyl chloride.

The hydrophobizing agent may for example involve contacting the RC coating layer with a fatty acid halide in a liquid, spray and/or vapor state.

Grafting of the fatty acid halide to the RC coating layer can be achieved by applying a fatty acid halide to the surface of the coating, followed by heating, which promotes the formation of covalent bonds between the fatty acid halide and the hydroxyl groups of the regenerated cellulose. Thus, in some embodiments the hydrophobization in step c) involves subjecting the fatty acid halide applied on the RC coating layer to heating in order to accelerate the formation of covalent bonds to the regenerated cellulose. In some embodiments, the heating is performed at a temperature of at least 100° C.

The inventors have further found that the advantageous properties of the coating, including water repellence and reduced surface roughness, can be further improved by subjecting the already hydrophobized coating layer to heat treatment. Thus, in some embodiments, the method further comprises a subsequent step d) heat treating the hydrophobized coating layer. In some embodiments, the heat treatment is performed at a temperature of at least 100° C. In some embodiments, the heat treatment comprises treatment with steam at a temperature of at least 100° C. The heat treatment may also be combined with a pressure treatment, e.g. calendaring or ironing.

Generally, while the products, polymers, materials, layers and processes are described in terms of “comprising” various components or steps, the products, polymers, materials, layers and processes can also “consist essentially of” or “consist of” the various components and steps.

While the invention has been described with reference to various exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A coated cellulose-based substrate, comprising
a cellulose-based substrate, and
a coating layer disposed on said cellulose-based substrate,
wherein said coating layer comprises regenerated cellulose and wherein said coating layer has been hydrophobized with a hydrophobizing agent.

2. The coated cellulose-based substrate according to claim 1, wherein the coating layer comprises at least 50 wt % of regenerated cellulose, based on a total dry weight of the coating layer.

3. The coated cellulose-based substrate according to claim 1, wherein the regenerated cellulose is obtained from dissolving pulp.

4. The coated cellulose-based substrate according to claim 1, wherein a dry basis weight of the coating layer is in a range of 0.1-30 gsm.

5. The coated cellulose-based substrate according to claim 1, wherein the hydrophobizing agent is covalently bound to the regenerated cellulose.

6. The coated cellulose-based substrate according to claim 1, wherein the hydrophobizing agent has
i) a hydrophobic portion and
ii) a reactive portion configured to form a covalent bond to the regenerated cellulose.

7. The coated cellulose-based substrate according to claim 1, wherein the hydrophobizing agent is selected from a group consisting of fatty acid halides, fatty acid anhydrides, epoxidized fatty acids, epoxidized fatty acid (methyl) esters, and combinations thereof.

8. (canceled)

9. (canceled)

10. The coated cellulose-based substrate according to claim 1, wherein the cellulose-based substrate is selected from a group consisting of: paper, paperboard, a bacterial cellulose membrane, a microfibrillated cellulose film, and combinations thereof.

11. (canceled)

12. The coated cellulose-based substrate according to claim 1, wherein a surface of said coating layer treated with the hydrophobizing agent has a water contact angle above 90°.

13. (canceled)

14. A method for manufacturing a coated cellulose-based substrate, said method comprising:
a) providing a cellulose-based substrate,
b) coating the cellulose-based substrate with a coating layer comprising a regenerated cellulose, and
c) hydrophobizing the coating layer with a hydrophobizing agent.

15. The method according to claim 14, wherein the coating layer is formed by applying cellulose dissolved in a solvent onto the cellulose-based substrate and treating the dissolved cellulose with a coagulation medium to obtain the coating layer comprising regenerated cellulose.

16. The method according to claim 15, wherein the solvent is a non-derivatizing solvent selected from a group consisting of an alkaline aqueous solution, N-methylmorpholine N-oxide (NMMO), an ionic liquid, and combinations thereof.

17. The method according to claim 15, wherein the solvent is a derivatizing solvent system selected from a group consisting of: urea and NaOH solution, thiourea, urea, and NaOH solution, and NaOH and carbon disulfide (CS2), and combinations thereof.

18. The method according to claim 14, wherein a dry basis weight of the coating layer is in the range of 0.1-30 gsm.

19. The method according to claim 14, wherein step b) further comprises drying the coated cellulose-based substrate.

20. The method according to claim 14, wherein the hydrophobizing agent is covalently bound to the regenerated cellulose.

21. The method according to claim 14, wherein the hydrophobizing agent has
i) a hydrophobic portion and
ii) a reactive portion configured to form a covalent bond to the regenerated cellulose.

22. The method according to claim 14, wherein the hydrophobizing agent is selected from a group consisting of fatty acid halides, fatty acid anhydrides, epoxidized fatty acids, epoxidized fatty acid (methyl) esters, and combinations thereof.

23. (canceled)

24. The method according to claim 14, further comprising the step
d) heat treating the hydrophobized coating layer.

25. The method according to claim 24, wherein the heat treating is performed at a temperature of at least 100° C.
